(19) United States
(12) Patent Application Publication
(10) Pub. No.: US 2025/0302759 A1
(43) Pub. Date: Oct. 2, 2025

(54) BIODEGRADABLE, CONTROLLED RELEASE MICROCAPSULES

(71) Applicant: ENCAPSYS, LLC, Appleton, WI (US)

(72) Inventors: Nianxi Yan, Appleton, WI (US); Sue M. Geiger, Freedom, WI (US)

(73) Assignee: ENCAPSYS, LLC, Appleton, WI (US)

(21) Appl. No.: 19/090,655

(22) Filed: Mar. 26, 2025

Related U.S. Application Data
(60) Provisional application No. 63/570,846, filed on Mar. 28, 2024.
(60) Provisional application No. 63/652,092, filed on May 27, 2024.

(51) Int. Cl. A61K 9/50; A61K 8/11; A61Q 19/00

(52) U.S. Cl. CPC A61K 9/5052; A61K 8/11; A61K 9/5089; A61Q 19/00; A61K 2800/10

(57) ABSTRACT

Biodegradable, controlled release microcapsules whose microcapsule walls comprise the reaction product of fractured plant proteins with isocyanates and/or bis- or poly-chloroformates.

### RELATED APPLICATIONS

The present application claims the benefit of prior U.S. Provisional Patent Application No. 63/570,846, filed Mar. 28, 2024, entitled “Biodegradable, Controlled Release Microcapsules” and No. 63/652,092, filed May 27, 2024, entitled “Biodegradable, Controlled Release Microcapsules”, the contents of both of which are hereby incorporated herein by reference in their entirety.

### FIELD

The present disclosure relates to biodegradable, controlled release microcapsules whose microcapsule walls comprise the reaction product of fractured plant proteins with isocyanates and/or bis- or poly-chloroformates. In particular and preferably the microcapsule walls are formed from fractured plant proteins and isocyanates wherein all or a majority of the isocyanates have at least two isocyanate groups.

### BACKGROUND

Microcapsules and microencapsulation technology are old and well known and their commercial applications varied. Microcapsules have played a significant role in various print technologies where a paper or other like substrate is coated with microcapsules containing ink or an ink-forming or inducing ingredient which microcapsules release the ingredient, generating an image, when fractured by pressure, as by a printing press or a stylus. Microcapsules have also played a significant role in various adhesive and sealant technologies including the encapsulation of solvents for solvent swellable/tackified preapplied adhesives whereby fracture of the microcapsules releases the solvent which softens or tackifies the adhesive to enable bonding and which re-hardened upon evaporation of the solvent. In other adhesive and sealant applications, the microcapsules contain one or more components of a curable or polymerizable adhesive or sealant composition which, upon release, leads to the cure or polymerization of the adhesive or sealant. In all of these early applications, functionality and efficacy, especially for long term storage and utility, is dependent upon the integrity of the microcapsule walls where the sought-after integrity pertains to both strengths, so as to avoid premature fracture, as well as impermeability, so as to prevent leakage and/or passage of the contents of the microcapsule through the microcapsule walls. In the former situation, parts having a preapplied microencapsulated adhesive have a tendency to bond together if they hit one another or are stacked upon one another where the pressure of the stack is sufficiently high. Even if not bonded, the fracture of the microcapsules results in less adhesive to effect the bond when the bond is intended. Similarly, if the microcapsule walls allow permeation of the active components through the cell wall, even a slow permeation, the product is short lived as cure will be effected when not intended.

As with most any technology, evolution of microencapsulation technology has led to many new applications, including applications that require changes in the physical properties of the microcapsules, especially their walls. New applications require microcapsules that fracture more readily, with less pressure, but not prematurely. Other applications require microcapsules that specifically allow for a controlled, slow release or permeation of the contents from within the microcapsules without the need to actually fracture the same. For example, perfume containing microcapsules are oftentimes applied to advertising inserts in magazines so that the reader can sample the smell of the perfume. Here strength is needed to avoid premature fracturing of the microcapsules due to the weight and handling of the magazine; yet, the microcapsules need ease of fracture so that the reader can simply scratch the treated area to release the contents of the microcapsule. At the same time, it is desirable to allow for some release of the contents, even without fracturing, to induce the reader to want to scratch the sample to get a more accurate sense of the smell.

Another application for microcapsules is in laundering and fabric treatments. A number of products exist wherein microcapsules of various ingredients, including perfumes, are applied to strips of a fabric material and added to the dryer wherein the tumbling action and/or heat of the dryer causes the microcapsules to fracture, releasing the ingredients which, in a volatilized state, permeate and deposit upon the contents of the dryer. This methodology applies that “fresh out of the dryer” smell, but is short lived as the perfume continues to volatilize from the treated fabric. Other products exist whereby microcapsules containing perfumes and other ingredients are applied directly or indirectly to the fabric, especially apparel, to provide a longer-lived freshness to the same. Here, the performance or efficacy of these products is oftentimes short lived as the content of the microcapsules escapes too readily from the microcapsules and/or the walls of the microcapsules are too weak and/or have too little give such that normal wearing of the fabric causes the microcapsules to break too readily.

Whether applications have driven the evolution of microcapsule technology or the evolution of microcapsule technology has driven their expanded applications, or perhaps a little of both, there has been and continues to be constant development in microencapsulation technology, both in terms of their production/process methodology and their chemistry. Early melamine formaldehyde microcapsules continue to evolve; yet concurrently, they have, to some extent, given way to acrylic and other microcapsule chemistries and technologies. In turn, both have continued to evolve further to dual walled microcapsules of each chemistry as well as both chemistries. While the basic building blocks of the capsule walls have largely remained the same, the specific selection of building blocks and methodology has led to newer and improved microcapsules enabling the microencapsulation of a broader array of ingredients, compounds and elements.

With all the advances and improvements noted and the continued expansion of microcapsule use for a myriad of applications, there is a growing buildup of microcapsules in the environment and, in following, in animal species feeding on materials containing and/or contaminated with microcapsules. Unfortunately, these microcapsules are formed of synthetic polymeric materials and remain in the environment for decades, if not centuries. Although not yet required by various governmental/regulatory agencies, movement is afoot to control the use of such polymeric microcapsules.

While capsules and, in some instances, microcapsules whose walls are formed of gelatin, albumin, polylactide and poly(lactide-co-glycolide), all generally considered biodegradable materials, have been used for a while in certain applications such as in the pharmaceutical and nutritional supplement industries, these materials are and their characteristics make them difficult to use, particularly in the production of microcapsules. Additionally, the resultant microcapsules are lacking in their physical properties and performance as compared to traditional, generally non-biodegradable, microcapsules, i.e., those prepared from petroleum-derived polymers. Microcapsules have also been formed of block animal based or derived polypeptides; however, again their properties and performance are limited: certainly not appropriate for the myriad of commercial applications of traditional, non-biodegradable microcapsules. Additionally, their method of production, solvent evaporation, and the limitations and difficulties with their use in coacervation processes, are generally not suitable or desirable for commercial large-scale production, let alone, encapsulation of the breadth of materials capable of being microencapsulated by more conventional microencapsulation techniques.

Hence, extensive efforts have been undertaken to develop microcapsule and microencapsulation methodologies whereby biodegradability is integrated into petroleum-based polymer microcapsules with the hope of mitigating the adverse impact thereof on the physical and performance properties of the resulting microcapsules. For example, Schwantes et. al., US 2023/0112578 A1, found beneficial microcapsules formed by the reaction of select gelatins with polyisocyanates. Similarly, Yan, US 2022/0162444 A1, established improved microcapsules through the use of low molecular weight peptides, those of 10,000 Da and less, as co-wall forming materials in combination with isocyanates and/or bis- or poly-chloroformates. Going to the extreme, Ehr et. al., US 2011/0045975 A1, employed amino acids themselves as the co-wall forming material.

Heretofore, however, these advancements in hybrid organic-petroleum based polymer microcapsules have been focused almost entirely on the use of gelatins, peptides, proteins and the like derived from animal sources owing to their water solubility and, hence, suitability and ease of use in typical oil-in-water based microencapsulation processes. This only makes sense as animal-based gelatins, peptides, proteins and the like have long been key additives and ingredients in the food and supplements industry and the industry, particularly the encapsulation industry, is comfortable with and extensively knowledgeable of their use, properties and performance. However, due, in large part, to the shift in diets affecting protein supplements and additives and the like, more attention in the food industry is now focusing on the use of plant-based gelatins, peptides, proteins and the like. Unfortunately, their use and the knowledge and expertise in relation thereto in the encapsulation industry is far less than with their animal counterparts. Indeed, it is found that their use in a number of applications is severely limited, if not impossible, due to their inherent water insolubility: an insolubility that, from a commercial perspective, makes them all but impossible and/or overly costly, to use in microencapsulation. Specifically, while some plant protein isolates have water solubility, the predominant component of such proteins are globulins which are water insoluble. Furthermore, certain studies have shown that while laboratory processes for the extraction and purification of plant proteins manifest some water solubility: the results are markedly different for those arising from commercial products and extraction processes. For example, water solubility of pea protein isolates prepared in the laboratory setting were found to have a water solubility of 66% whereas the same extracts from commercial settings had water solubility of only 5% (Cited in Ma, K K, et. al., “Functional Performance of Plant Proteins,” Foods, 2022, 11, 594—doi.org/10.3390/foods11040594). In large volumes, it is not commercially feasible to prepare suitable plant proteins for use in the commercial production of microcapsules.

Despite all the advances and continued efforts to establish microcapsules having improved biodegradability without compromising the properties thereof, there is still a continuing need and urgency for further developments owing to the myriad of applications for microcapsules and the different end-use applications in which they are found and the different environments and environmental factors they must contend with. Similarly, there is a continued need for improved performance and properties, especially enhanced biodegradability, with reasonable, preferably reduced, costs. Furthermore, there is a growing desire for overcoming the limitations and obstacles associated with the poor water solubility, if not insolubility, of plant proteins in order to expand their use in microencapsulation where both biodegradability of the organic biological component and performance/physical properties of the petroleum based component are retained, if not improved.

### SUMMARY

According to the present teachings there are provided novel microcapsules and methods of forming the same, wherein the microcapsule walls comprise the reaction product of plant proteins, particularly those plant proteins that, absent the fragmentation of the present teaching, have poor water solubility or are water insoluble, with conventional cross-linkers, most especially isocyanates and/or bis- or poly-chloroformates. Specifically, there are provided novel microcapsules and methods of forming the same, wherein the microcapsule walls comprise the reaction product of fragmented plant proteins with isocyanates and/or bis- or poly-chloroformates, wherein the fragmentation of the plant proteins results in protein isolates whose median particle size, i.e., D50, is preferably reduced by at least about 40%, more preferably at least about 50%, most preferably at least about 60% from the median particle size of the protein isolate prior to fragmentation. Preferred fragmented proteins have a median particle size that is from about 0.1% to about 60%, more preferably from about 5% to about 35%, of the median particle size of the plant protein isolate prior to fragmentation.

Fragmentation of the plant proteins may be achieved by a number of methodologies including mechanical shear such as homogenization, ultrasound, pulverization and the like; pH degradation; high temperature degradation; solvent degradation; salt degradation; and combinations thereof.

Property and performance characteristics of the microcapsules can be controlled by selection of size and type of proteins, the isocyanate and/or bis- or poly-chloroformate, the microencapsulation process employed and the weight ratio of isocyanate and/or bis- or poly-chloroformate to the fragmented protein. Typically, the weight ratio of the former, particularly the isocyanate, to the fragmented protein is from 100:1 to 1:100, preferably from 50:1 to 1:50, more preferably 10:1 to 1:10. While such higher ratios are suitable, it is especially preferred that the weight ratio of the former to the fragmented protein be from 1:5 to 1:0.2, preferably 1:4 to 1:0.5, more preferably 1:2 to 1:1.

### BRIEF DESCRIPTION OF THE FIGURES

FIG. 1 is a bar graph of fragrance release from fragmented soy protein/isocyanate microcapsules wherein the fragmentation was a result of homogenization by a shear mixer.

FIG. 2 is a bar graph of fragrance release from fragmented soy protein/isocyanate microcapsules wherein the fragmentation was a result of ultrasonication.

### DETAILED DESCRIPTION

The present teaching is directed to novel microcapsules and the process by which they are made. In particular, the present teaching is directed to microcapsules that are biodegradable and serve as carriers for various core materials contained therein including solids, hydrophilic agents, hydrophobic agents, lipophilic agents and the like. Most especially, the present teaching is directed to carrier microcapsules that are biodegradable and have controlled release properties for a liquid/volatile core material contained therein.

The microcapsules according to the present teaching are unique in that their walls comprise the reaction product of i) fragmented plant proteins, especially plant proteins that, in the absence of the fragmentation of the present teaching, manifest, poor water solubility and/or are water insoluble, with ii) one or more conventional (for the encapsulation/microencapsulation industry) cross-linker. Exemplary crosslinkers include one or more isocyanates, especially di- and/or poly-functional isocyanates, bis- and/or poly-chloroformates, acid chlorides, sulfonyl chlorides, polyfunctional alcohols, and combinations thereof: such cross-linkers may be employed in the form of monomers, oligomers, and/or polymers/pre-polymers. In following, though not to be limited thereto, the following description is presented with a focus on the preferred cross-linkers, namely the isocyanates, especially diisocyanates and/or polyisocyanates; the bis- and/or poly-chloroformates; and combinations thereof. Fragmentation of the plant proteins typically and preferably results in plant protein isolate compositions whose median particle size, i.e., D50, is preferably reduced by at least about 40%, more preferably at least about 50%, most preferably at least about 60%, from the median particle size of the protein isolate prior to fragmentation. Preferred fragmented proteins have a median particle size that is from about 0.1% to about 60%, more preferably from about 5% to about 35%, of the median particle size of the plant protein isolate prior to fragmentation.

Similarly, the process by which the present microcapsules are prepared is unique in that it calls for the use of fragmented plant proteins together with the aforementioned conventional cross-linkers, most especially the isocyanates, especially diisocyanates and/or polyisocyanates, and the bis- and/or poly-chloroformates and combinations thereof, as the wall forming materials. The microencapsulation process may be a water-in-oil process, an oil-in-water process or a water-in-oil-in water process: the latter process is especially useful when the core material is hydrophilic or water soluble/dispersible and one wants to avoid the use of large volumes of oil phase materials as is required of the water-in-oil process. For convenience, the following description is presented in terms of the oil-in-water process: though those skilled in the art will readily appreciate and acknowledge the adaptability of the process to a water-in-oil and water-in-oil-in-water process.

Suitable diisocyanates and polyisocyanates for use in the practice of the present teaching include those known for use in the formation of polyurethane, polyurea, and polyurethane-urea microcapsules and are well known to those of ordinary skill in the art. These isocyanates include aliphatic, cycloaliphatic, aromatic, polyaromatic, etc., isocyanates as well as combinations thereof. Such isocyanates typically have from 2 to 16, preferably 4 to 10 carbon atoms in the basic hydrocarbon skeleton. While the diisocyanates are preferred, polyisocyanates, especially those having 3 or 4 cyanato (NCO) groups, 3 to 10 cyanato groups in the case of dimers and oligomers, as well as combinations of diisocyanates and polyisocyanates are also desirable and useful. While a single isocyanate is suitable, it is also desirable to employ combinations of isocyanates, e.g., a combination of di- and/or poly-isocyanates, a combination of aliphatic and aromatic isocyanates, as well as combinations of both. In this regard, the weight percent of each isocyanate may be from 0-100% of the combination. In the case of combinations of aliphatic and aromatic isocyanates, it is preferred that each be present in an amount of at least 5% by weight, preferably at least 10% by weight, more preferably at least 20% by weight, depending upon the desired leakage rate. For example, aliphatic/aromatic isocyanate microcapsules with lower leakage will typically have at least 50% by weight, more preferably at least 70% by weight, of the aliphatic isocyanate. Further, while mono-isocyanates may be present, the di- or poly-isocyanates comprise at least 50% by weight, preferably at least 70% by weight, more preferably at least 80% by weight, most preferably at least 90% by weight of the isocyanate component. Preferably the isocyanate is a di-isocyanate or a combination of di- and poly-isocyanates wherein at least 50% by weight, more preferably at least 70% by weight, most preferably at least 85% by weight of the isocyanate is a di-isocyanate.

Exemplary aliphatic and cycloaliphatic isocyanates include 2,2,4-trimethylhexamethylene diisocyanate, 2,4,4-trimethylhexamethylene diisocyanate,1,4-tetramethylene diisocyanate, 1,6-hexamethylene diisocyanate (HDI), isophorone diisocyanate IPDI), 4,4′-methylene-bis-(cyclohexane diisocyanate), cylcohexane-1,4-diisocyante, and adducts, trimers, biurets, symmetric trimers, asymmetric trimers thereof, especially of hexamethylene diisocyanate, and the like, including trimethyol propane adducts of hexamethylene diisocyanate.

Aromatic isocyanates include m- and p-tetramethylxylene diisocyanate (TMXDI), α,α′-xylylene diisocyanate, methylene diisocyanate (MDI), especially 4,4′-diphenylmethane diisocyanate, toluene diisocyanate (TDI), 1,4-phenylene diisocyanate, 1,3-phenylene diisocyanate, 2,4,6-triisocyanate toluene, 4,4′,4′-triisocyanate triphenyl methane, 1,2,4-benzene triisocyanate, 2,5-norbornene diisocyanate, 3,3′-dimethyldiphenyl-4,4′-diisocyanate, naphthalene diisocyanate, and adducts, trimers, biurets, symmetric trimers, asymmetric trimers thereof, especially of toluene diisocyanate and methylene diisocyanate, and the like. Exemplary trimers include, but are not limited to, the trimer of hexamethylene diisocyanate sold under the trademark Desmodur® N-3390 by Bayer Corporation of Pittsburgh, Pa. and the trimer of isophorone diisocyanate.

Suitable isocyanates also include oligomeric and low molecular weight polymeric isocyanates: again, materials that are well known to those of ordinary skill in the art. Preferably, in keeping with the desirability of degradability, such oligomeric and low molecular weight polymeric isocyanates are formed of an isocyanate and low molecular weight polyols, preferably C₁ to C₆ polyols, especially diols and/or triols, most especially linear diols and triols. Exemplary oligomeric and low molecular weight polymeric isocyanates include, but are not limited to, trimethylol propane adducts of the isocyanates, especially those of toluene diisocyanate, methylene diisocyanate, and xylylene diisocyanate.

Alternatively or in addition to the isocyanates, one may employ bis- and/or poly-chloroformates as the co-reactant with the fragmented proteins to form the polyurethane microcapsules. Such chloroformates are well known and widely available as well and generally have the structure ClCO—U—OCCl wherein the U is hydrocarbyl, e.g., alkylidene, or an oxygen containing hydrocarbyl linking group, e.g., diethylene glycol bis chloroformate. Exemplary bischloroformates include monoethylene glycol bis-(chloroformate), diethylene glycol bis(chloroformate), butanediol bis(chloroformate), hexanediol bis(chloroformate), neopentyldiol bis(chloroformate), bisphenol A bis(chloroformate) and mixtures thereof.

The critical component of the wall forming materials is the fragmented plant proteins. Specifically, the present teaching allows for the use of plant proteins, including, especially, those having high globulin content and/or which manifest poor water solubility and/or are water insoluble in the absence of fragmentation, as presently taught, and, heretofore, have been unsuitable for use in the production, particularly the commercial scale production, of microcapsules, especially in the production of microcapsules formed of both biological organic components and petroleum-based components, most especially in oil-in-water processes. Not intending to be bound by theory, it is believed that the fractionation of the plant proteins makes reactive groups more available and/or helps unfold the globulin structure.

Generally speaking, any of the known plant protein extracts and/or isolates may be employed as the source of the fragmented proteins. Suitable sources include, but are not limited to, soy protein, pea protein, chickpea protein, beans protein, lentils protein, potato protein, wheat protein, oats protein, malt protein, rye protein, barley protein, rice protein, algae protein, gluten protein, lupin protein, other legume proteins, and mixtures thereof. These proteins may be in the form of protein concentrates, protein isolates, or a combination thereof. Such proteins are widely available commercially and include food or pharmaceutical grade and non-food grades depending upon the end-use application for the resulting microcapsules. Generally speaking, plant proteins are considered insoluble if their solubility in water at room temperature and neutral pH is less than 25%. Poorly soluble plant proteins will generally have a solubility at those conditions of from 25% to less than 70%. It is recognized that certain isolates or fractions of such proteins have varying solubility, particularly under different conditions; but, the marked benefit of the present disclosure relates especially, though not exclusively, to the protein isolates/-extracts, not the individual fractions; though, those too will benefit from the present teaching and fragmentation of such fractions is contemplated and within the scope of the present invention and teaching, especially those fractions whose solubility parameters fall within the foregoing ranges as well. In following, reference herein to fragmented plant proteins includes fragmented extracts as well as fragmented isolates: the latter having already undergone some fractionation.

Fragmentation of the plant proteins may be achieved by any of the known methods for breaking protein chains, with or without unfolding the chains. Such methodologies include mechanical shear such as homogenization, ultrasonication, pulverization and the like; pH degradation; chemical degradation such as glycation, phosphorylation, acylation, etc.; high temperature degradation; solvent degradation; salt degradation; and combinations thereof. Exemplary fragmentation methods are further described in Grossman, L., et., “Current insights into protein solubility: A review of its importance for alternative proteins,” Food Hydrocolloids, 137 (2023) 108416, the contents of which are incorporated herein by reference. Preferably, the fragmentation process involves several of the foregoing parameters, more preferably one in which one of the parameters is a mechanical shear. Most preferably, the fragmentation process involves a mechanical shear together with pH and/or temperature control/modification for a specified period time or duration of fragmentation.

As noted above, the fragmented protein isolates employed in the practice of the present teaching typically comprise protein fragment compositions having a median particle size, i.e., D50, which is at least about 40% less, more preferably at least about 50% less, most preferably at least about 60% less, than the median particle size of the protein isolate prior to fragmentation. Preferred fragmented proteins have a median particle size that is from about 0.1% to about 60%, more preferably from about 5% to about 35%, of the median particle size of the plant protein isolate prior to fragmentation. The ultimate particle size and distribution thereof is both a factor of the fragmentation process used and its duration as well as the desired properties of the resulting microcapsules. As shown below, besides allowing for the use of previously unsuitable plant proteins in microcapsule production, the present process also enables one to custom design the properties, especially release properties, of the resultant microcapsules.

As discussed in further detail below, to aid in solubilizing and/or distributing the wall forming components, especially the fragmented proteins, in the aqueous phase, one may also employ surfactants and other solubilizing/dispersing aids. Suitable surfactants and solubilizing/dispersing aids include polyvinyl alcohol (PVA), polystyrene sulfonate (PSS), carboxymethylcellulose (CMC), sodium salt of naphthalene sulfonate condensate, and the like, as well as mixtures thereof.

As noted, the microcapsules are formed by the reaction of the fragmented proteins with one or more isocyanates and/or bis- or poly-chloroformates. Preferred isocyanates are the di- and/or poly-isocyanates, as discussed above, although mono-isocyanates may also be used. However, when the isocyanate component includes mono-functional isocyanates, at least 50% by weight, preferably at least 70%, more preferably at least 80% by weight, most preferably at least 90% by weight of the isocyanate component is a di- and/or higher isocyanate. Typically, the weight ratio of the isocyanate and/or bis- or poly-chloroformate to the fragmented protein is from 100:1 to 1:100, preferably from 50:1 to 1:50, more preferably 10:1 to 1:10. While such higher ratios are suitable, it is especially preferred that the weight ratio of the former to fragmented protein be from 1:5 to 1:0.2, preferably 1:4 to 1:0.5, more preferably 1:2 to 1:1.

Core materials that may be encapsulated in accordance with the present teaching include a myriad of substances, consistent with those materials that are encapsulated by existing technologies and chemistries. Core materials include solid particles, semi-solid materials, hydrophilic liquids, lipophilic liquids, hydrophobic liquids, volatile liquids, and the like. Specific selection depends upon the intended utility of the microcapsules. Indeed, microcapsules have a myriad of applications across various industries and consumer products including, but not limited to, agrochemicals, pharmaceuticals, cosmetics industry, personal care products, laundering detergents, homecare & cleaning products, oral care, dental care, textiles, paper, mining, oil industry, water treatment, adhesives, coatings, coatings, plastics, sealants, construction, paints, inks and dye formulations. Exemplary core materials include, but are not limited to UV reflectors, UV absorbers, pigments, dyes, colorants, scale inhibitors, emollient oils, insecticides, detergents, printing inks, corrosion and rust, recording materials, inhibitors, antioxidants, pour point depressants, catalysts, initiators, waxes, deposition inhibitors, dispersants, flame retardants, biocides, active dye tracer materials, silicone conditioners, shampoos, biocides, adhesives, anti-fouling agents, odor control agents, cosmetic additives, oxidizing agents, personal care actives, agrochemicals, fertilizers, fats, nutrients, enzymes, liquid crystals, natural oils, fragrances, flavor and perfume oils, crop protection agents, medicaments, pharmaceuticals, phase change materials and the like. Specific examples of core materials are disclosed in, e.g., US 2013/0337023, U.S. Pat. Nos. 10,456,766, 8,119,214, 9,714,397, 10,485,739, 4,977,060, 10,675,277, US20070138673, and US20130302392, all of which are hereby incorporated by reference, among a myriad of other patents, patent publications and the like.

The following presents a non-limiting list of exemplary core materials.

Linear or branched hydrocarbons of different chain lengths and viscosities such as mineral oil, petrolatum, white oil (also known as paraffin oil), dodecane, isododecane, squalane, hydrogenated polyisobutylene, polybutene, polydecene, docosane, hexadecane, isohexadecane and other isoparaffins, which are branched hydrocarbons.

Alcohol, diol, triol or polyol esters of carboxylic or dicarboxylic acids, of either natural or synthetic origin having straight chain, branched chain and aryl carboxylic acids include diisopropyl sebacate, diisopropyl adipate, isopropyl myristate, isopropyl palmitate, myristyl propionate, cetyl lactate, myristyl lactate, lauryl lactate, C12-15 alkyl lactate, dioctyl malate, decyl oleate, isodecyl oleate, ethylene glycol distearate, ethylhexyl palmitate (octyl palmitate), isodecyl neopentanoate, tridecyl neopentanoate, castoryl maleate, isostearyl neopentanoate, di-2-ethylhexyl maleate, cetyl palmitate, myristyl myristate, stearyl stearate, cetyl stearate, isocetyl stearate, dioctyl maleate, octyl dodecyl stearate, isocetyl stearoyl stearate, octyldodecyl stearoyl stearate dioctyl sebacate, diisopropyl adipate, cetyl octanoate, glyceryl dilaurate, diisopropyl dilinoleate and caprylic/capric triglyceride. Naturally occurring includes triglycerides, diglycerides, monoglycerides, long chain wax esters and blends of these. Examples of naturally derived ester-based oils and waxes include, but are not limited to, argan oil, corn oil, castor oil, coconut oil, cottonseed oil, menhaden oil, avocado oil, beeswax, carnauba wax, cocoa butter, palm kernel oil, palm oil, peanut oil, shea butter, jojoba oil, soybean oil, rapeseed oil, linseed oil, rice bran oil, pine oil, sesame oil, sunflower seed oil and safflower oil. Also useful are hydrogenated, ethoxylated, propoxylated and maleated derivatives of these materials, e.g., hydrogenated safflower oil, hydrogenated castor oil. Cholesterol and its esters and derivatives, as well as natural materials comprising cholesterol derivatives such as lanolin and lanolin oil.

Phospholipids (e.g., lecithin), sphingophospholipids, ceramides and related materials.

C4-C20 alkyl ethers of polypropylene glycols, C1-C20 carboxylic acid esters of polypropylene glycols, and di-C8-C30 alkyl ethers. Also included are PPG-14 butyl ether, PPG-15 stearyl ether, diodyl ether, dodecyl octyl ether, and mixtures thereof.

Saturated and unsaturated fatty acids including but not limited to oleic, palmitic, isostearic, stearic, ricinoleic, linoleic and linolenic acid. Carboxylic monoesters and polyesters of sugars (mono-, di- and polysaccharides) and related materials.

Silicones such as polyalkylsiloxanes, polydialkylsiloxanes, polydiarylsiloxanes, and polyalkarylsiloxanes may also be used. This includes the polydimethylsiloxanes, which are commonly known as dimethicones. Further cyclic siloxanes (e.g., cyclopentasiloxane) and dimethiconoles, alkyl methicones, alkyl dimethicones, dimethicone copolyols, amino-functional silicones (e.g., amodimethicone, trimethylsilyloxyamodimethicone) and amphoteric silicones (e.g., cetyl PEG/PPG-15/15 butyl ether dimethicone, and bis-PEG-18 methyl ether dimethyl silane).

Oily and oil-soluble extracts of plant materials such as flowers and herbs. This comprises a wide range of materials, with some non-limiting examples including extracts of rosemary, green, white or black tea, orchid, grape seed, sage, soybean, echinacea, amica, rosehip, olive, artichoke. Further plant-extracted oil-soluble components such as lycopene and other mixed carotenoids, capsaicin and capsaicinoids, polyphenols (e.g., rosmarinic acid), terpenes and terpenoids, oleoresins.

Exemplary dyes include, but are not limited to, Green 6 (CI 61570), Red 17 (CI 26100), Violet 2 (CI 60725) and Yellow 11 (CI 47000). Examples of oil-dispersible pigments include, but are not limited to Beta Carotene (CI 40800), Chromium Hydroxide Green (CI 77289), Chromium Oxide Green (CI 77288), Ferric Ferrocyanide (CI 77510), Iron Oxides (CI 77491, 77492 77499), Pigment Blue 15 (CI74160), Pigment Green 7 (CI 74260), Pigment Red 5 (CI 12490), Red 30 (CI 73360), Titanium Dioxide (CI 77891) and Ultramarines (CI 77007).

Exemplary pharmaceutical active, especially for dermatological treatment of conditions of skin, hair and nails include, but is not limited to, topical anaesthetics, anti-fungal, anti-bacterial, anti-viral, anti-dandruff, anti-acne and anti-inflammatory agents (steroidal and non-steroidal).

Examples of vitamin and derivatives include tocopherol, tocopheryl acetate, retinol, retinyl palmitate, ascorbyl palmitate, niacinamide, beta carotene.

Fragrances suitable for use in the practice of the present teaching include without limitation, any combination of perfumes, flavors, essential oils, sensates and plant extract or mixture thereof that is capable of being encapsulated in accordance with the present application. A list of suitable fragrances is provided in U.S. Pat. Nos. 4,534,891, 5,112,688, 5,145,842, 6,194,375, 20110020416 and PCT application Nos. WO2009153695 and WO2010/044834 and Perfumes Cosmetics and Soaps, Second Edition, edited by W. A. Poucher, 1959. Each of the foregoing documents is incorporated herein by reference in its entirety.

Typical representative perfume and sensate components include, but are not limited to, linalool, coumarin, geraniol, citral, limonene, citronellol, eugenol, cinnamal, cinnamyl alcohol, benzyl salicylate, menthol, menthyl lactate, eucalyptol, thymol, methyl salicylate, methylfuran, menthone, cinnamaldehyde.

Typical representative examples for essential oils include, but are not limited to, orange, lavender, peppermint, lemon, pine, rosemary, rose, jasmine, tea tree, lemon grass, bergamot, basil, spearmint, juniper, clove, aniseed, fennel, cypress, fir, black pepper, sandalwood, cedarwood, rosewood, cardamom, cinnamon, coriander, eucalyptus, geranium, ginger, chamomile, grapefruit, neroli, petitgrain, thyme, vetiver and ylang ylang.

Non-limiting examples of phase change materials include n-octacosane, n-heptacosane, n-hexacosane, n-pentacosane n-tetracosane, n-tricosane, n-docosane, n-heneicosane, n-eicosane, n-nonadecane, n-octadecane, n-heptadecane, n-hexadecane, n-pentadecane, n-tetradecane and n-tridecane.

Chemical and physical sunscreens/UV filters, e.g., 3-Benzylidene Camphor, 4-Methylbenzylidene camphor, Aminobenzoic acid (PABA)′ Avobenzone, Benzophenone 4 (Sulisobenzone), Benzophenone 5, Benzophenone 8, Benzophenone-3, Benzylidene camphor sulfonic acid, Bis-ethylhexyloxyphenol methoxyphenol triazine (Escalol S), Butyl methoxy dibenzoylmethane, Camphor benzalkonium methosulfate, Cinoxate, Diethylamino hydroxybenzoyl hexyl benzoate, Dioxybenzone, Disodium phenyl dibenzimidazole tetrasulfonate, Drometrizole trisiloxane, Ensulizole, Ethylhexyl dimethyl PABA, Ethylhexyl methoxycinnamate, Ethylhexyl salicylate, Ethylhexyl triazone, Homosalate, Isoamyl p-methoxycinnamate, Meradimate, Menthyl anthranilate, Methylene bis-benzotriazolyltetramethylbutylphenol/Bisoctrizole (Tinosorb M), Octocrylene, Octinoxate, PEG-25 PABA, Octisalate, Oxybenzone, Padimate O, Phenylbenzimidazole sulfonic acid, Polyacrylamidomethyl Benzylidene Camphor, Polysilicone-15, TEA-salicylate, Terephthalylidene dicamphor sulfonic acid, Titanium dioxide, Trolamine Salicylate and zinc oxide.

Hair treatment materials, other than those covered in the previous ingredient list. This includes cationic conditioning agents comprising tertiary and quaternary amino groups (e.g., quaternium-70, quaternium-80, stearamidopropyl dimethylamine, behentrimonium methosulfate, dicocodimonium chloride, dicetyldimonium chloride, distearyldimonium chloride hydroxyethyl cetyldimonium phosphate). Further, UV and color protectants (e.g., dimethylpabamidopropyl laurdimonium tosylate), heat protectants and styling polymers (e.g., vinyl pyrrolidone and vinylcaprolactam derivatives, such as PVP vinyl Caprolactam/DMAPA Acrylates Copolymer).

Consumer and agrichemical ingredients include insecticides and insect repellants, including, N,N-Diethyl-meta-toluamide, IR3535, Icaridin, Picaridin, Saltidin, Citronella, Permethrin, Neem oil and Lemon Eucalyptus.

Core materials also include polymeric materials, especially oil-soluble polymeric materials, which have film-forming properties on skin and hair, such as VP/Hexadecene Copolymer, Tricontanyl PVP and VP/Eicosene Copolymer as well as cosmetic and personal care actives, which are used for the conditioning or cosmetic treatment of skin, hair or nails are listed extensively and typically covered in IP.com publications IPCOM000128968D published 23 Sep. 2005 and IPCOM000133874D published 13 Feb. 2006, the contents of which are hereby incorporated by reference.

Core materials also include corrosion inhibitors which may be selected from the group consisting of carboxylic acids and derivatives such as aliphatic fatty acid derivatives, imidazolines and derivatives; including amides, quaternary ammonium salts, rosin derivatives, amines, pyridine compounds, trithione compounds, heterocyclic sulfur compounds, quinoline compounds, or salts, quats, or polymers of any of these, and mixtures thereof. For example, suitable inhibitors include primary, secondary, and tertiary monoamines; diamines; amides; polyethoxylated amines, diamines or amides; salts of such materials; and amphoteric compounds. Still other examples include imidazolines having both straight and branched alkyl chains, phosphate esters, and sulfur containing compounds.

Similarly, core materials include lipophilic scale inhibitors including those based on phosphate esters, and polyacrylates as well as oxidizing agents including inorganic or organic peroxides such as calcium peroxide, magnesium peroxides and lauryl peroxides.

As noted above, it may be desirable, if not necessary, to employ processing aids to assist in the production of the microcapsules. Two areas where processing aids are especially beneficial is in the solubilization/dispersion of the fragmented proteins in the water phase and in the dispersion or emulsification of the oil phase in the water phase or, if applicable, the dispersion or emulsification of the water phase in the oil phase. Generally speaking, the use of such processing aids is most beneficial where the core material is difficult to mill, particularly where it is otherwise difficult to obtain the target or desired particle/droplet size during the emulsification process. Preferred processing aids are emulsifiers and surfactants.

Emulsifiers of all types are suitable for use in the practice of the present process though it is to be appreciated, and those skilled in the art will readily recognize, that different systems, e.g., different fragmented proteins, oil phases and core materials, will be better suited with one or more classes of emulsifiers than others. Specifically, while the present teachings are applicable to anionic, cationic, non-ionic and amphoteric emulsifiers generally, preferred emulsifiers are the cationic and non-ionic emulsifiers, particularly those having polyalkylether units, especially polyethylene oxide units, with degrees of polymerization of the alkylene ether unit of greater than about 6. Preferred emulsifiers are those which significantly reduce the interfacial tension between the continuous water phase and dispersed oil phase composition, and thereby reduce the tendency for droplet coalescence. In this regard, generally the emulsifiers for use in the water phase for aiding in the oil in water emulsion or dispersion will have HLB values of from 11 to 17.

Exemplary emulsifiers include, but are not limited to polyvinyl alcohols, including PVA itself and especially those polyvinyl alcohols that are partially hydrolyzed; cellulose derivatives such as ethyl hydroxyethyl cellulose, 2-hydroxyethyl cellulose, hydroxybutyl methylcellulose, hydroxypropyl methylcellulose, etc.; gums such as acacia gum and xantham gum; poly(meth)acrylic acids and derivatives; and poly(styrene-co-maleic acid) and derivatives; and the like. Most preferably, the emulsifier/emulsion stabilizer is a polyvinyl alcohol, particularly a polyvinyl alcohol that has been derived from polyvinyl acetate, wherein between 85 and 95%, preferably 88 to 90% of the vinyl acetate groups have been hydrolyzed to vinyl alcohol units.

Additional exemplary anionic surfactants and classes of anionic surfactants suitable for use in the practice of the present teaching include: sulfonates; sulfates; sulfosuccinates; sarcosinates; alcohol sulfates; alcohol ether sulfates; alkylaryl ether sulfates; alkylaryl sulfonates such as alkylbenzene sulfonates and alkylnaphthalene sulfonates and salts thereof; alkyl sulfonates; mono- or di-phosphate esters of polyalkoxylated alkyl alcohols or alkylphenols; mono- or di-sulfosuccinate esters of C₁₂ to C₁₅ alkanols or polyalkoxylated C₁₂ to C₁₅ alkanols; ether carboxylates, especially alcohol ether carboxylates; phenolic ether carboxylates; polybasic acid esters of ethoxylated polyoxyalkylene glycols consisting of oxybutylene or the residue of tetrahydrofuran; sulfoalkylamides and salts thereof such as N-methyl-N-oleoyltaurate Na salt; polyoxyalkylene alkylphenol carboxylates; polyoxyalkylene alcohol carboxylates alkyl polyglycoside/alkenyl succinic anhydride condensation products; alkyl ester sulfates; naphthalene sulfonates; naphthalene formaldehyde condensates; alkyl sulfonamides; sulfonated aliphatic polyesters; sulfate esters of styrylphenyl alkoxylates; and sulfonate esters of styrylphenyl alkoxylates and their corresponding sodium, potassium, calcium, magnesium, zinc, ammonium, alkylammonium, diethanolammonium, or triethanolammonium salts; salts of ligninsulfonic acid such as the sodium, potassium, magnesium, calcium or ammonium salt; polyarylphenol polyalkoxyether sulfates and polyarylphenol polyalkoxyether phosphates; and sulfated alkyl phenol ethoxylates and phosphated alkyl phenol ethoxylates; sodium lauryl sulfate; sodium laureth sulfate; ammonium lauryl sulfate; ammonium laureth sulfate; sodium methyl cocoyl taurate; sodium lauroyl sarcosinate; sodium cocoyl sarcosinate; potassium coco hydrolyzed collagen; TEA (triethanolamine) lauryl sulfate; TEA (Triethanolamine) laureth sulfate; lauryl or cocoyl sarcosine; disodium oleamide sulfosuccinate; disodium laureth sulfosuccinate; disodium dioctyl sulfosuccinate; N-methyl-N-oleoyltaurate Na salt; tristyrylphenol sulphate; ethoxylated lignin sulfonate; ethoxylated nonylphenol phosphate ester; calcium alkylbenzene sulfonate; ethoxylated tridecylalcohol phosphate ester; dialkyl sulfosuccinates; perfluoro (C₆-C₁₈)alkyl phosphonic acids; perfluoro(C₆-C₁₈)alkyl-phosphinic acids; perfluoro(C₃-C₂₀)alkyl esters of carboxylic acids; alkenyl succinic acid diglucamides; alkenyl succinic acid alkoxylates; sodium dialkyl sulfosuccinates; and alkenyl succinic acid alkylpolyglycosides. Further exemplification of suitable anionic emulsifiers include, but are not limited to, water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate (SDS), alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, alkylene-maleic anhydride copolymers such as isobutylene-maleic anhydride copolymer, or ethylene maleic anhydride copolymer gum arabic, sodium alginate, carboxymethylcellulose, cellulose sulfate and pectin, poly(styrene sulfonate), pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; maleic anhydride copolymers (including hydrolyzates thereof), polyacrylic acid, polymethacrylic acid, acrylic acid alkyl acrylate copolymers such as acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxymodified polyvinyl alcohol, sulfonic acid-modified polyvinyl alcohol and phosphoric acid-modified polyvinyl alcohol, phosphated or sulfated tristyrylphenol ethoxylates.

Exemplary amphoteric and cationic emulsifiers include alkylpolyglycosides; betaines; sulfobetaines; glycinates; alkanol amides of C₈ to C₁₈ fatty acids and C₈ to C₁₈ fatty amine polyalkoxylates; C₁₀ to C₁₈ alkyldimethylbenzylammonium chlorides; coconut alkyldimethylaminoacetic acids; phosphate esters of C₈ to C₁₈ fatty amine polyalkoxylates; alkylpolyglycosides (APG) obtainable from an acid-catalyzed Fischer reaction of starch or glucose syrups with fatty alcohols, in particular C₈ to C₁₈ alcohols, especially the C₈ to C₁₀ and C₁₂ to C₁₄ alkylpolyglycosides having a degree of polymerization of 1.3 to 1.6., in particular 1.4 or 1.5. Additional cationic emulsifiers include quaternary ammonium compounds with a long-chain aliphatic radical, e.g., distearyldiammonium chloride, and fatty amines. Among the cationic emulsifiers which may be mentioned are alkyldimethylbenzylammonium halides, alkyldimethylethyl ammonium halides, etc. specific cationic emulsifiers include palmitamidopropyl trimonium chloride, distearyl dimonium chloride, cetyltrimethylammonium chloride, and polyethyleneimine. Additional amphoteric emulsifiers include alkylaminoalkane carboxylic acids betaines, sulphobetaines, imidazoline derivatives, lauroamphoglycinate, sodium cocoaminopropionate, and the zwitterionic emulsifier cocoamidopropyl betaine.

Suitable non-ionic emulsifiers are characterized as having at least one non-ionic hydrophilic functional group. Preferred non-ionic hydrophilic functional groups are alcohols and amides and combinations thereof. Examples of non-ionic emulsifiers include: mono and diglycerides; polyarylphenol polyethoxy ethers; polyalkylphenol polyethoxy ethers; polyglycol ether derivatives of saturated fatty acids; polyglycol ether derivatives of unsaturated fatty acids; polyglycol ether derivatives of aliphatic alcohols; polyglycol ether derivatives of cycloaliphatic alcohols; fatty acid esters of polyoxyethylene sorbitan; alkoxylated vegetable oils; alkoxylated acetylenic diols; polyalkoxylated alkylphenols; fatty acid alkoxylates; sorbitan alkoxylates; sorbitol esters; C₈ to C₂₂ alkyl or alkenyl polyglycosides; polyalkoxy styrylaryl ethers; amine oxides especially alkylamine oxides; block copolymer ethers; polyalkoxylated fatty glyceride; polyalkylene glycol ethers; linear aliphatic or aromatic polyesters; organo silicones; polyaryl phenols; sorbitol ester alkoxylates; and mono- and diesters of ethylene glycol and mixtures thereof; ethoxylated tristyrylphenol; ethoxylated fatty alcohol; ethoxylated lauryl alcohol; ethoxylated castor oil; and ethoxylated nonylphenol; alkoxylated alcohols, amines or acids; amides of fatty acids such as stearamide, lauramide diethanolamide, and lauramide monoethanolamide; long chain fatty alcohols such as cetyl alcohol and stearyl alcohol; glycerol esters such as glyceryl laurate; polyoxyalkylene glycols and alkyl and aryl ethers of polyoxyalkylene glycols such as polyoxyethylene glycol nonylphenyl ether and polypropylene glycol stearyl ether. Polyethylene glycol oligomers and alkyl or aryl ethers or esters of oligomeric polyethylene glycol are preferred. Also preferred as non-ionic emulsifiers are polyvinyl alcohol, polyvinyl acetate, copolymers of polyvinyl alcohol and polyvinylacetate, carboxylated or partially hydrolyzed polyvinyl alcohol, methyl cellulose, various latex materials, stearates, lecithins, and various surfactants. It is known that polyvinyl alcohol is typically prepared by the partial or complete hydrolysis of polyvinyl acetate. Accordingly, by reference to polyvinyl alcohol we intend to include both completely and partially hydrolyzed polyvinyl acetate. With respect to the latter, it is preferred that the polyvinyl acetate be at least 50 mole % hydrolyzed, more preferably, at least 75 mole % hydrolyzed.

Where the emulsifier is a polymeric emulsifier, especially one having or derived from an acrylic ester, e.g., a polyacrylate, the molecular weight is generally at least 10,000, preferably at least 20,000, most preferably 30,000 or more. Additionally, the amount of emulsifier is typically from about 0.1 to about 40% by weight, more preferably from about 0.2 to about 15 percent, most preferably from about 0.5 to about 10 percent by weight based on the total weight of the formulation. It is to be appreciated that certain acrylic polymers and copolymers may perform both as an emulsifier as well as a polymerizable and/or non-polymerizable component in forming the microcapsule wall. With respect to the latter, the polymeric emulsifier, particularly those in the nature of higher molecular weight polymers, are trapped and/or incorporated into the polymer wall as it is formed. This is especially likely where the nature of the water phase changes and the solubilized polymer comes out of solution.

Though not required, it may be desirable to employ other stabilizing substances that may be used, alone or in combination with the aforementioned materials, including ionic monomers. Typical cationic monomers include dialkyl amino alkyl acrylate or methacrylate including quaternary ammonium or acid addition salts and dialkyl amino alkyl acrylamide or methacrylamide including quaternary ammonium or acid addition salts. Typical anionic monomers include ethylenically unsaturated carboxylic or sulphonic monomers such as acrylic acid, methacrylic acid, itaconic acid, allyl sulphonic acid, vinyl sulphonic acid especially alkali metal or ammonium salts. Particularly preferred anionic monomers are ethylenically unsaturated sulphonic acids and salts thereof, especially 2-acrylamido-2-methyl propane sulphonic acid, and salts thereof.

Similarly, though not necessary, the water phase compositions and the core phase compositions may further contain other ingredients conventional in the art including, e.g., chain transfer agents and/or agents which help control the molecular weight/degree of polymerization of the wall forming monomer, thereby aiding in the movement of the oligomer/prepolymer through the respective oil phase and water phase compositions. Suitable chain transfer agents include, but are not limited to, lower alkyl alcohols having from 1 to 5 carbon atoms, mercaptoethanol, mercaptopropanol, thioglycolic acid, isooctylmercaptoproprionate, tert-nonylmercaptan, pentaerythritol tetrakis(3-mercaptoproprionate), dodecylmercaptan, formic acid, halogenated hydrocarbons, such as bromoethane, bromotrichloromethane, or carbon tetrachloride, and the sulfate, bisulfate, hydrosulfate, phosphate, monohydrogen phosphate, dihydrogen phosphate, toluene sulfonate, and benzoate salts of sodium and potassium, especially sodium hypophosphite and sodium bisulfate. If present, the chain transfer agents are preferably used in amounts ranging from 0.01 to 5%, preferably from 0.5 to 3%, by weight with respect to the wall forming monomers and/or oligomers employed.

Following on the foregoing, the wall forming composition may also include various polyfunctional amines and alcohols which can be dispersed or dissolved in water or an aqueous solution and are capable of reacting with the isocyanate and/or chloroformate, especially the isocyanate, to serve as cross-linkers for modifying the microcapsule wall physical properties. In particular, the preferred cross-linkers can be employed to manipulate or control release characteristics while having minimal or modest impact upon degradability. Such cross-linking agents are well known in the art and generally have two or more, preferably two to five, primary or secondary amine groups or hydroxy groups or a combination of hydroxy and amine groups, including, e.g., aldehydes and their derivatives, epoxies and their derivatives, isothiocyanates and their derivatives, etc. Generally, they may be individual compounds, dimers, oligomers or low molecular weight polymers. Most especially, they tend to be lower molecular weight, generally having molecular weights of 500 or less, preferably 250 or less. Exemplary amines include 1,2-ethylenediamine, 1,3-diamino propane, 1,4-diaminobutane, 1,6-diaminohexane, hydrazine, 1,4-diaminocyclohexane, 1,3-diamino-1-methylpropane, diethylenetriamine, triethylenetetramine, tetraethylene-pentamine, bis(2-methylamino-ethyl) methylamine, triethanolamine, bis(dimethylamino-ethyl) ether, triisopropanolamine, ethanolamine, guanidine amine and its derivatives, etc. Exemplary polyols include ethylene glycol, propylene glycol, diethylene glycol, dipropylene glycol, triethylene glycol, 2-ethylhexanediol-1,3 glycerin, 1,2,6-hexane triol, trimethylol propane, trimethylol ethane, and tris(hydroxy-phenyl)propane. If present, these cross-linking agents are preferably used in amounts ranging from 0.01 to 20%, preferably from 0.5 to 10%, by weight with respect to the wall forming monomers and/or oligomers employed. These cross-linkers may be added to the water phase prior to formation of the emulsion or to the emulsion once formed.

The particle size of the microcapsules of the present teaching will vary widely depending upon the core material as well as the intended end-use application, and the constraints of the method by which the microcapsules, especially the core material dispersion/emulsion, is formed. Typically, the volume weighted median particle size will range from about 2 microns to about 200 microns, preferably from about 5 microns to about 50 microns, most preferably from about 10 microns to about 20 microns.

In following, the thickness of the microcapsule walls is likewise dependent upon the intended end-use of the microcapsules and is influenced, as well, by the wall forming materials themselves and the physical properties of/desired of the microcapsule walls. Wall thickness is generally controlled by the amount of wall forming materials employed in the encapsulation process as well as the size/quantity of the beads/droplets of the dispersed phase to be encapsulated. Generally speaking, the wall forming materials comprise from 3 to 30, preferably from 5 to 20, more preferably from 8 to 15, weight percent of the microcapsule forming materials, i.e., wall forming materials and core materials.

Microencapsulation of the core material with the wall forming materials may be attained through any of the known methods for microencapsulation. Suitable techniques include coacervation, interfacial polymerization, air suspension, centrifugal extrusion, spray drying, pan coating, in-situ polymerization, and by forming a dispersion of core material and shell material and applying a pressure shock wave to the dispersion as described in Redding Jr. (U.S. Pat. No. 5,271,881, incorporated herein by reference). The specific selection of the method and the materials depends upon the nature, including the physical state and/or chemistry, of the material to be encapsulated, e.g., whether the carrier material is in a liquid form or a solid, semi-solid or gel-like particulate form. Exemplary methods are set forth in the following paragraphs as well as in, fo^{r} example, Schwantes (U.S. Pat. No. 6,592,990), Nagai et. al. (U.S. Pat. No. 4,708,924). Baker et. al. (U.S. Pat. No. 4,166,152), Wojciak (U.S. Pat. No. 4,093,556), Matsukawa et. al. (U.S. Pat. No. 3,965,033), Matsukawa (U.S. Pat. No. 3,660,304), Ozono (U.S. Pat. No. 4,588,639), Irgarashi et. al. (U.S. Pat. No. 4,610,927), Brown et. al. (U.S. Pat. No. 4,552,811), Scher (U.S. Pat. No. 4,285,720), Shioi et. al. (U.S. Pat. No. 4,601,863), Kiritani et. al. (U.S. Pat. No. 3,886,085), Jahns et. al. (U.S. Pat. Nos. 5,596,051 and 5,292,835), Matson (U.S. Pat. No. 3,516,941), Chao (U.S. Pat. No. 6,375,872), Foris et. al. (U.S. Pat. Nos. 4,001,140; 4,087,376; 4,089,802 and 4,100,103), Greene et. al. (U.S. Pat. Nos. 2,800,458 and 2,730,456), Clark (U.S. Pat. No. 6,531,156), Saeki et. al. (U.S. Pat. Nos. 4,251,386 and 4,356,109), Hoshi et. al. (U.S. Pat. No. 4,221,710), Hayford (U.S. Pat. No. 4,444,699), Hasler et. al. (U.S. Pat. No. 5,105,823), Stevens (U.S. Pat. No. 4,197,346), Riecke (U.S. Pat. No. 4,622,267), Greiner et. al. (U.S. Pat. No. 4,547,429), and Tice et. al. (U.S. Pat. No. 5,407,609), among others and as taught by Herbig in the chapter entitled “Encapsulation” in Kirk Othmer, Encyclopedia of Chemical Technology, V. 13, Second Edition, pages 436-456 and by Huber et. al. in “Capsular Adhesives”, TAPPI, Vol. 49, No. 5, pages 41A-44A, May 1966, all of which are incorporated herein by reference.

Generally speaking, the first step in the encapsulation process is the preparation of discrete particles, domains, droplets, or beads of the core material. Where such materials are in solution or liquid form and the encapsulation is to be by way of, e.g., coacervation, interfacial polymerization, etc., the solution or liquid containing the core material is subjected to high shear mixing or agitation to create a suspension, emulsion or colloidal system of discrete domains of the core material of the requisite size. Where the core material is a heat sensitive material, e.g., a wax or wax-like material, the carrier, with the therein incorporated core material, is heated above its melt temperature and then subjected to a similar high shear mixing or agitation in a liquid medium, preferably one of the wall forming materials or the phase material, e.g., water or oil phase, to create discrete droplets of the core material and then cooled to allow the solid particles to form, before encapsulating. Where the core material is a solid or substantially solid material, it may be ground and sorted to the desired particle size before encapsulation. Such methods, as well as additional alternative methods for preparation of the particles or discrete domains for encapsulation are widely used in industry and well known to those skilled in the art.

Although not limited thereto, the microcapsules according to the present teaching are preferably prepared by interfacial polymerization. Here, the core material is solubilized, dispersed or emulsified in a liquid solution of one of the materials to be used as/containing one of the wall forming materials, preferably the oil phase comprising the isocyanate, or, in the case of a water-in-oil-in-water system, in a first water phase, preferably free of wall forming material, which water phase containing the core material is then dispersed or emulsified in the oil phase which, in turn, is dispersed or emulsified in a second water phase, which is ultimately the continuous phase for the interfacial polymerization and contains the other wall forming material, namely the fragmented protein.

As noted above, the microcapsules made in accordance with the present teaching have a myriad of commercial and consumer applications across most all industries and in many commercial products.

In one embodiment, microcapsules described herein may be incorporated in personal care products and compositions including, but not limited to, cosmetics, drug delivery systems, hair care products, skin treatment products and oils, pharmaceuticals, pigment dispersions, preservative compositions, skin coloring products, skin restorative products, styling products for hair, sunscreen and suntan lotions, sprays, oils, creams and the like, water proof/resistance products, wear resistance products and additives, shower gels, shampoos, and thermal protecting/enhancing compositions. Dental personal care compositions include denture adhesives, toothpastes, mouth washes, chewing gums and the like.

The microcapsules may be used in numerous pharmaceutical applications and compositions including peroral and topical dosage forms, such as tablets, pellets, capsules, dermatological products (creams, gels, ointments, sprays, lotions, and foams), transdermal patches and the like.

The microcapsules may also be used in conjunction with a myriad of agrochemicals, especially those listed extensively in U.S. Pat. No. 5,389,688, to ISP, which is incorporated herein by reference in its entirety.

In common consumer products, the microcapsules may be used to incorporate actives such as fabric conditioners, liquid laundering detergents, powdered laundering detergents, dish washing detergents, hard surface cleaners, anti-static agents, anti-odor agents, antimicrobial agents, etc., into various household cleaners and other “cleaning” products such as air fresheners, sprays, and the like, as well as onto textiles, paper and the like as surface modifiers or coatings.

The microcapsules of the teaching can be advantageously used in controlling perfume release in fragrant consumer products. With the microcapsules of the present teaching there is a considerable improvement in longevity and intensity of the encapsulated perfume in actual use. Examples of consumer products comprising perfume microcapsules according to certain aspects of the present application may fall into product group categories of laundering detergents, cosmetics, personal care products, dish washing detergents and house cleaners. More specific examples of consumer products include fabric conditioners, liquid/powdered laundering detergents, dish washing detergents, hair shampoos, hair conditioners, hair styling gels, soaps, body washes, shower gels, all-purpose cleaners including hard surface cleaners, carpet cleaners, body lotions, antiperspirant/deodorants and spray-able products.

In following, in another embodiment of the present teaching there is provided a method for producing fragrance loaded microcapsules with improved substantivity for incorporation into, (i) laundry detergents; (ii) fabric softener compositions; and (iii) drier-added fabric softener articles, these when deposited on fabrics during laundry treatment and capable of remaining on the textile following initial application and which is capable of later being sheared by the application of mechanical force. Accordingly, the encapsulated fragrance provides a “burst” of fragrance during wear and/or cleaning due to breakage of the capsule wall.

Alternatively, the fragrance microcapsules of the present application can be formulated into solid fabric care compositions with polysaccharides such as sugars according to the procedure described in US Patent No 2011/0082066, the contents of which are hereby incorporated by reference. The solid fabric care products can be used for delivering fragrances onto the textile articles during the washing/cleaning cycle and subsequently the laundered textiles have beneficial fragrance odor profile during the wear.

Alternatively, the fragrance microcapsules can be incorporated in 2-in-1 powdered detergent and conditioner compositions according to the processes described in U.S. Pat. Nos. 4,698,167 and 5,540,850 and also crystalline laundry additives as described in the US application 2011/97369 and PCT WO 2010/000558, which are incorporated herein by reference.

For some embodiments, it may be preferred to incorporate a plurality of core materials into a single microcapsule and/or provide mixtures of such microcapsules, For example, one may add one or more preservatives and/or antimicrobial agents in the delivery matrix in addition to the respective actives, such as, but not limited to, benzoic acid, sorbic acid, dehydroacetic acid, piroctone olamine, DMDM hydantoin, IPBC, triclosan, bronopol, isothiazolinones, parabens, phenoxyethanol, and combination thereof.

In another embodiment, the core material may be a phase change material or mixtures thereof for temperature control. Typical phase change materials exhibit a melting temperature from −20° C. to 100° C. and generally comprise linear or branched hydrocarbons or fatty esters or mixtures thereof, of different chain lengths and melting points. The microcapsules containing the phase change material core material can be coated or sprayed onto or incorporated into suitable materials including textile fibers, such as cotton and polyester, during the spinning process or coated directly onto textiles or incorporated into building construction material for example bricks, gypsum, and the like, to allow for temperature control by use of latent heat of fusion.

It is further contemplated that microcapsules of the present teaching can be used in the construction industry in conjunction with cements, plaster boards, breeze blocks, chipboards, heat transfer fluids, sealants, adhesives etc. In this instance, the core material can be a phase change material, biocide, flame retardant, catalyst, epoxy resin, etc.

The present microcapsules also have a number of automotive applications including the use of encapsulated phase change materials in the coolant systems, the use of encapsulated lubricant additives such as anti-wear additives in engine oils, and the use of encapsulated UV absorbers and/or anti-corrosive agents for car coatings.

The microcapsules described herein may also be used in conjunction with additives used in plastics such as flame retardants, catalysts, pigments, light stabilizers, UV absorbers, and the like, all of which can be encapsulated to allow higher compatibilities, longevity and self-healing of the plastic material. For example, the core material can be a catalyst for self-healing, an UV absorber for protection from photodamage due to UV light, or a thermochromic material for color change in coatings across a broad spectrum of industry.

The microcapsules according to the present teaching may also be used in oilfield applications. Here the microcapsules may contain traditional oilfield chemicals such as corrosion inhibitors, scale inhibitors, oxidizing agents, crosslinking agents, catalysts, acidizing agents, biocides, demulsifiers, enzymes, polymers, lubricants, shale inhibitors, solvents, and surfactants. The encapsulated oil field chemicals can be applied advantageously at the different petroleum extraction stages from drilling, cementing, stimulation to production and enhanced oil recovery. The release mechanisms of delivery of the oilfield chemical can be by temperature, dilution, pH and shear at the relevant points of applications.

### EXAMPLES

### Preparation of Fragmented Proteins

Two fragmentation methods were employed to make the fragmented proteins. The first was by way of homogenization using a Polytron PT 6100 high-speed homogenizer from Kinematica AG of Malters, Switzerland. The second was by sonication using a VCX 750 ultrasonic processor from Sonics & Materials Inc., of Newtown, CT. In each method, a protein dispersion was prepared in a 1-liter stainless steel reactor by dispersing 20.34 grams of soy protein isolate (MP Biomedicals, LLC of Irvine, CA) in 345.8 grams of deionized water with a mixer at 400 rpm at room temperature and the pH adjusted using a 10% caustic soda solution to attain the pH as indicated in the specific examples below. The particle size of the dispersed protein was then determined using a LA-350 Particle Size Distribution Analyzer from Horiba Instruments Inc., of Arbor, MI., with particle size being reported as median size D50 and/or D90. Once the starting particle size was determined, the mixer was replaced with the homogenizer or ultrasonic processor, as appropriate, with samples taken at different time intervals and measured for particle size, again using the LA-350 Particle Size Distribution Analyzer. In each case, an initial study, designated “−1”, was performed to assess the effect and results of the fragmentation. The two methods were repeated, “−2” designation, each on a plurality of identical dispersions containing 13.6 grams of the soy protein in 217 grams of deionized water, each dispersion subjected to the given fragmentation method for a given period of time and/or until a similar, though different, temperature was attained in the first, i.e., the −1 series of experiments, and the particle size determinations made and the samples set aside for preparation of microcapsules.

### Homogenization-1

Once the starting particle size was determined, the mixer was replaced with the Polytron PT 6100 high-speed homogenizer with a PT-DA 6045/6T aggregator running as 20,000 rpm. The temperature of the dispersion increased with time due to energy input from the homogenizer. Fragmentation was performed at both neutral pH (7.34) and a basic pH (11.33). Samples of the fragmented protein dispersion were taken at various temperatures and the particle size (D50) determined as presented in Table 1.

TABLE 1
Homogenization - 2

| | | | Protein Size D50, |
|---|---|---|---|
| pH | Homogenizer RPM | Temp. ° C. | um |
| 7.34 | mixing, 400 | 22 | 132 |
| | 20k | 53 | 68.1 |
| | 20k | 75 | 21.9 |
| | 20k | 90 | 23.2 |
| | 20k | 95 | 13.6 |
| 11.33 | mixing, 400 rpm | 22 | 132 |
| | 20k | 53 | 55.2 |
| | 20k | 75 | 35.0 |
| | 20k | 90 | 35.8 |
| | 20k | 95.0 | 10.7 |

Table 2 presents the particle size distribution of the homogenizer fragmented proteins used in the preparation of microcapsules.

TABLE 2
Sonication - 1

| | Protein Size D50, | Protein Size D90, |
|---|---|---|
| Sample | um | um |
| Control | 132 | 278 |
| 1 | 19.2 | 103.5 |
| 2 | 17.2 | 38.3 |
| 3 | 13.9 | 37.5 |
| 4 | 8.4 | 16.5 |

Once the starting particle size was determined, the mixer was replaced with the VCX 750 ultrasonic processor with a probe of a half-inch in diameter operating at a frequency of 20 kHz and an amplitude of 80%. Again, the temperature of the dispersion increased with time due to energy input from the ultrasound treatment. Fragmentation was performed at both neutral pH (7.34) and a basic pH (11.33). Samples of the fragmented protein dispersion were taken at various times and the particle size (D50) determined as presented in Table 3.

TABLE 3
Sonication - 2

| | | | Protein Size, D50, |
|---|---|---|---|
| pH | Time (min) | Temp. C. | um |
| 7.32 | 0 | 22 | 114 |
| | 4 | 50 | 38.3 |
| | 5 | 75 | 48.7 |
| | 12 | 90 | 33.9 |
| 11.39 | 0 | 22 | 114 |
| | 3 | 50 | 94.8 |
| | 4 | 75 | 8.37 |
| | 5 | 90 | 7.65 |

Table 4 presents the particle size distribution of the sonication fragmented proteins used in the preparation of microcapsules.

TABLE 4
Microencapsulation

| | Protein Size D50, |
|---|---|
| Sample | um |
| Control | 130 |
| 1 | 23.18 |
| 2 | 5.68 |
| 3 | 0.45 |

A number of microcapsules were formed in accordance with the present teaching to show the ability of the formation of microcapsules whose walls are formed of both plant proteins and petroleum-based monomer/polymer. In those instances where the fragmented proteins were readily dispersible/soluble in the aqueous phase, no emulsifier was used: in those instances where dispersion/solubility was less than desired, an emulsifier was added. Typically, the microcapsules were formed in accordance with the following steps using non-water soluble core materials:
An oil phase solution was prepared by mixing the core material, 81.4 grams of a fragrance blend comprising a combination of odiferous compounds including benzyl acetate, isobornyl acetate, hexyl salicylate and the like, with 81.4 grams of Captex® 355 caprylic/capric triglyceride from ABITEC Corporation of Columbus, OH (as the fragrance diluent) in a beaker at room temperature.
Once the core material was thoroughly mixed, 6.78 of Mondur® MR light, an aromatic polymeric isocyanate based on diphenylmethane-diisocyanate (MDI), from Covestro LLC of Pittsburgh, PA, was added and the mixture mixed.
The foregoing oil phase solution was then dispersed in reactor vessels containing the unfragmented and fragmented protein solutions noted above, under high shear milling: the high shear milling was maintained until the targeted emulsion droplet size of approximately 10 microns was attained.
Once the targeted droplet size was attained, milling with the milling blade was paused and removed and a mixing blade inserted as its replacement in order to keep the emulsion mixed.
Thereafter, the temperature of the reaction vessel was raised from ambient temperature to 50° C. over a period of 60 minutes and the temperature then maintained at 50° C. for another 60 minutes. Thereafter, the temperature was raised to 65° C. over a period of 60 minutes and then maintained at that temperature for another 120 minutes. Finally, the temperature of the reaction mix was subsequently raised to 85° C. over a period 60 minutes and maintain at that temperature for an additional 6 hours at which time the encapsulation process was deemed completed.
Upon completion, the resulting microcapsule slurry was allowed to cool to ambient temperature and found to be of low viscosity and generally uniform and smooth with no visible thickening and/or agglomeration.

The foregoing process was followed to produce a plurality of batches of microcapsules, one for each of the fragmented soy proteins identified in Tables 2 and 4 above. Soy protein was selected due to its partial water solubility in order to demonstrate the benefits of the fragmented protein over an unfragmented protein that has limited wall forming capabilities.

Notwithstanding the steps noted above, it is also to be appreciated that somewhat elevated temperatures may be desirable to aid in quicker and easier formation of the solutions and/or the emulsion. Furthermore, one may vary the milling temperature and/or the rate of temperature ramp up during wall formation depending on the reactivity and quantity of the raw materials used. In this respect, it is to be appreciated that the capsule slurry may become gelled, or emulsion become coalesced during temperature ramp up and/or wall formation if too high of a milling temperature and/or too fast a rate of temperature ramp up are used. In this respect, milling at room temperature is preferred if the raw materials are very reactive. On the other hand, higher milling temperature is preferred if the raw materials are less reactive.

Given the organic fragmented protein segments, biodegradability of the microcapsules is believed inherent; however, from a commercial utility perspective, the greater concern relates to the integrity of the capsule walls and their release characteristics.

### Test Method: Release of Fragrance

The test method employed to evaluate fragrance release is based upon the CIPAC MT190. Here, the capsule slurry was placed into a 110 ml jar and 100 ml of hexane with an internal standard (dicyclohexyl phthalate) subsequently added. The jar was placed on a horizontal roller, rolling at 70 RPM. Thereafter, 1 ml aliquots of the solutions were removed at time 0, 15 minutes, 30 minutes, 60 minutes and 180 minutes, and placed into an injection vial whose contents were then analyzed via GC-FID for the core of interest.

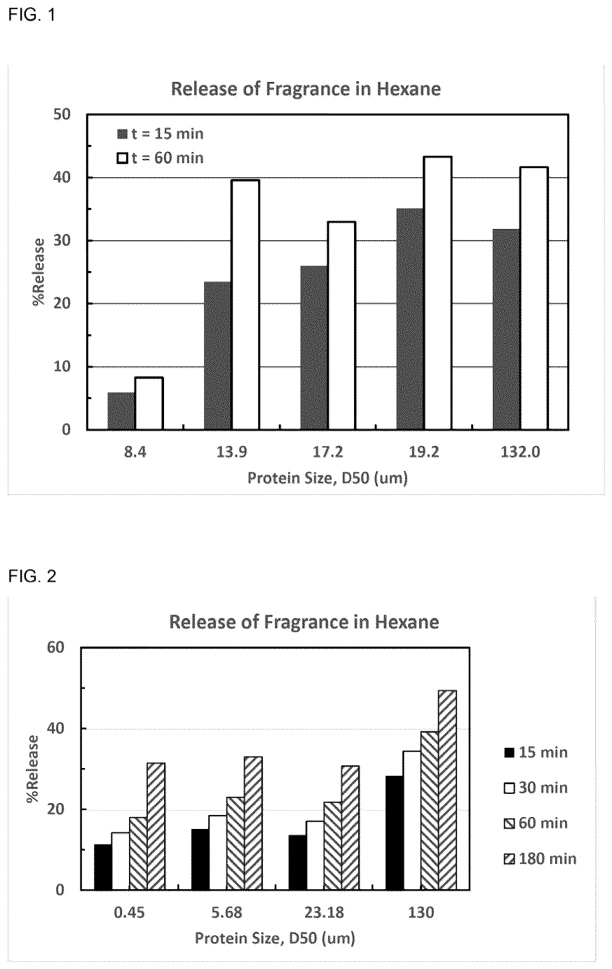

The results attained for the microcapsules formed with the homogenized fragmented soy proteins are presented in Table 5 and FIG. 1 and the results for the microcapsules form with the sonically fragmented soy proteins are presented in Table 6 and FIG. 2.

TABLE 5

| Sample | % Release of Fragrance (min) | | | | |
|---|---|---|---|---|---|
| Table 2 | 0 | 15 | 30 | 60 | 180 |
| Control | 0 | 31.82 | 36.05 | 41.66 | 52.14 |
| 1 | 0 | 35.11 | 40.13 | 43.3 | 55.58 |
| 2 | 0 | 26.00 | 30.04 | 32.95 | 40.55 |
| 3 | 0 | 23.48 | 31.24 | 39.58 | 71.81 |
| 4 | 0 | 5.91 | 7.05 | 8.25 | 13.04 |

TABLE 6

| Sample | % Release of Fragrance (min) | | | | |
|---|---|---|---|---|---|
| Table 4 | 0 | 15 | 30 | 60 | 180 |
| Control | 0 | 28.24 | 34.36 | 39.16 | 49.35 |
| 1 | 0 | 13.61 | 17.01 | 21.8 | 30.68 |
| 2 | 0 | 15.0 | 18.41 | 22.99 | 32.94 |
| 3 | 0 | 11.32 | 14.14 | 17.99 | 31.38 |

The results shown in Tables 5 and 6 demonstrate the ability to establish and control release properties of the microcapsules, essentially create custom release microcapsules, based upon the degree/extent of fragmentation. Further control and “dialing in” of release properties can be attained by combining different fragmented proteins and/or using less or more of the same plant protein fragment. In this respect, once a small sample/library establishing the correlation between the select plant protein fragment size, preferably the median particle of the fragmented sample, e.g., the D50, and release properties, one can then customize the fragment size, especially the median fragment size, for a given release rate.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. No unclaimed language should be deemed to limit the invention in scope. Any statements or suggestions herein that certain features constitute a component of the claimed invention are not intended to be limiting unless reflected in the appended claims.

The principles, preferred embodiments, and modes of operation of the present invention have been described in the foregoing specification. The invention which is intended to be protected herein, however, is not to be construed as limited to the particular forms disclosed, since these are to be regarded as illustrative rather than restrictive variations and changes can be made by those skilled in the art without departing from the spirit and scope of the invention.

We claim:

1. A microcapsule comprising the reaction product of one or more conventional cross-linkers with one or more fragmented plant proteins which has been subjected to fragmentation whereby its median particle size D50 has been reduced by at least about 40% from its mean particle size prior to fragmentation.

2. The microcapsule of claim 1 wherein the cross-linker is one or more di-isocyanates and/or poly-isocyanates and/or bis- and/or poly-chloroformates.

3. The microcapsule of claim 1 wherein the cross-linker is one or more aromatic and/or aliphatic di-isocyanates and/or poly-isocyanates.

4. The microcapsule of claim 1 wherein the plant protein is an extract or an isolate of a plant protein which is characterized as being poorly soluble or insoluble in water prior to fragmentation.

5. The microcapsule of claim 1 wherein the median particle size of the plant protein has been reduced by at least about 50%.

6. The microcapsule of claim 1 wherein the median particle size of the plant protein has been reduced by at least about 60%.

7. The microcapsule of claim 1 wherein the median particle size of the fragmented plant protein is from about 0.1% to about 60% of the median particle size of the plant protein isolate prior to fragmentation.

8. The microcapsule of claim 1 wherein the median particle size of fragmented plant protein is from about 5% to about 35% of the median particle size of the plant protein isolate prior to fragmentation.

9. The microcapsule of claim 1 wherein the weight ratio of the cross-linker to the fragmented protein is from 100:1 to 1:100.

10. The microcapsule of claim 1 wherein the weight ratio of the cross-linker to the fragmented protein is from 50:1 to 1:50.

11. The microcapsule of claim 1 wherein the weight ratio of the cross-linker to the fragmented protein is from 10:1 to 1:10.

12. The microcapsule of claim 1 wherein the weight ratio of the cross-linker to the fragmented protein is from 1:5 to 1:0.2.

13. The microcapsule of claim 1 wherein the weight ratio of the cross-linker to the fragmented protein is from 1:4 to 1:0.5.

14. The microcapsule of claim 1 wherein the weight ratio of the cross-linker to the fragmented protein is from 1:2 to 1:1.

15. A method of forming microcapsules comprising forming 1) an emulsion or dispersion of water phase composition and an oil phase, the water phase composition comprising one or more fragmented plant proteins which has been subjected to fragmentation whereby its median particle size D50 has been reduced by at least about 40% from its mean particle size prior to fragmentation and the oil phase containing a conventional cross-linking agent reactive with the plant protein, and 2) subjecting the emulsion to conditions suitable for effecting the polymerization and cross-linking of the plant protein and the cross-linking agent and allowing the reaction to continue for sufficient time to effect the formation of the microcapsules.

16. The method of claim 15 wherein the cross-linker is one or more di-isocyanates and/or poly-isocyanates and/or bis- and/or poly-chloroformates.

17. The method of claim 15 wherein the cross-linker is one or more di-isocyanates and/or poly-isocyanates.

18. The method of claim 15 wherein the plant protein is an extract or an isolate of a plant protein which is characterized as being poorly soluble or insoluble in water prior to fragmentation.

19. A method of forming a microcapsule of a predetermined biodegradability and/or release rate comprising forming 1) an emulsion or dispersion of water phase composition and an oil phase, the water phase composition comprising one or more fragmented plant proteins and the oil phase containing a conventional cross-linking agent reactive with the plant protein, and 2) subjecting the emulsion to conditions suitable for effecting the polymerization and cross-linking of the plant protein and the cross-linking agent and allowing the reaction to continue for sufficient time to effect the formation of the microcapsules wherein the fragmented plant protein is selected to have a mean particle size in the range of from 0.1% to 60% of the original particle size corresponding to the desired release properties.

20. The method of claim 19 wherein the cross-linker is one or more di-isocyanates and/or poly-isocyanates and/or bis- and/or poly-chloroformates.
